(19) United States
(12) Patent Application Publication
(10) Pub. No.: US 2026/0220771 A1
(43) Pub. Date: Jul. 30, 2026

(54) A METHOD AND SYSTEM THAT CAN BE USED TO ADAPT THE MARKINGS OF DEEP LEARNING SYSTEMS USED FOR OBTAINING ANATOMICAL IMAGES FOR MEDICAL PURPOSES IN ACCORDANCE WITH THE MOVEMENT, CHANGE IN THE IMAGE AND CHANGES IN PROCESSING SPEED

(71) Applicant: SMART ALFA TEKNOLOJI SANAYI VE TICARET ANONIM SIRKETI, Ankara (TR)

(72) Inventors: Ahmet Sahin YENER, Ankara (TR); Ayhan OKUYAN, Ankara (TR); Ömer Ferhad SARIOGLU, Ankara (TR); Utku KAYA, Ankara (TR)

(21) Appl. No.: 19/143,575

(22) PCT Filed: Dec. 28, 2023
(86) PCT No.: PCT/TR2023/051818

§ 371 Date: Jun. 26, 2025

(30) Foreign Application Priority Data
Dec. 28, 2022 (TR) 2022/020874

(51) Int. Cl. G06T 7/00; G06T 7/246; G06V 10/82; G06V 20/70; G16H 50/20

(52) U.S. Cl. CPC G06T 7/0012; G06T 7/248; G06V 10/82; G06V 20/70; G16H 50/20; G06T 2207/20081; G06T 2207/20084; G06V 2201/031

(57) ABSTRACT

The present invention relates to the method and system developed to adapt the markings of deep learning systems that mark the images created with methods (especially ultrasonographic or elastographic methods) used for obtaining anatomical images for medical purposes in accordance with the movement, change and changes in processing speed in the image and to increase the clinical stability of deep learning system markings.

### TECHNICAL FIELD

The present invention relates to the method and system developed to adapt the markings of deep learning systems that mark the images created with methods (especially ultrasonographic or elastographic methods) used for obtaining anatomical images for medical purposes in accordance with the movement, change and changes in processing speed in the image and to increase the clinical stability of deep learning system markings.

### STATE OF THE ART

Deep learning is one of the most popular methods used in medical anatomical imaging analysis. The use of deep learning with algorithms with different network architectures is fairly common in the field of health, especially in medical anatomical images. These methods are frequently used in areas such as early diagnosis, early treatment of diseases, reducing the intensity of experts, reducing the cost of imaging, and different expert opinions.

The detection and marking of anatomical structures on the medical anatomical image with methods such as artificial intelligence is actually a prediction made by artificial intelligence for the flowing image of the application in a single scene.

Marking can be done on a single frame, and the processing power is based on machines processing a single frame. Singular-frame-processing algorithms have been selected to benefit from them since the literature and resources are generally single-frame-processing machines. Therefore, at least a part of the image frames created in the flowing image during imaging will need to be followed.

Video-based structures should be used to make sense of ultrasound images, and the form of ultrasound imaging is fused with the video structure. Singular-frame-processing machines can create estimates that are incompatible with the displayed anatomical structures or previous estimates.

Current ultrasound devices are formed with peripheral equipment such as a computer unit and an imaging unit. Ultrasound devices can be operated at a constant processing speed of 20 FPS in most devices as an industry standard. However, deep learning systems used to interpret the image are operated on this peripheral equipment.

However, the change in image frames during imaging may vary according to the movement/movement speed of the scanner (ultrasound probe, etc.) of the imaging device used, the movement of the patient and the processing speed of the peripheral equipment used. Since these systems generally aim to reduce or reduce the need for qualified experts, they are expected to give as stable responses as possible in different environments and different situations.

Today, very different imaging medical devices equipped with peripheral equipment with different structural features (processor power, etc.) are in use on the market. It may be possible to equip these existing devices with deep learning systems that mark the created images.

In this case, if the moving image is created by the system, it may be possible to receive different reactions from different devices. For example, the fact that devices with different technologies reveal different FPS (frames per second) outputs may affect the operation of deep learning systems that mark the images.

Deep learning systems operating with single frames used in medical imaging systems are not affected by the flow rate; however, since the visualization of reactions cannot be done through video, spacing these reactions can pose an important technical problem.

Similarly, keeping the scanner of the imaging device constant or moving it at different speeds will affect the adaptation of the visualizations to the image, as it will change the rate of change in the image frames.

For this purpose, systems aiming to measure the movement between consecutive images are used in the current art. These systems are operated to use the information created in the video to edit the marking elements.

These systems aim to follow the image flowing through every pixel in the image. This application may be inadequate because it requires a serious processor load and gives inefficient results in certain situations.

For example, when said systems are used in ultrasound applications, they do not work due to noise.

The existing systems develop by selecting the points that can be followed up in the image and successfully in order to follow up more accurately after the failure to follow up for every pixel in the image.

However, in these newly developed systems, inadequate results may occur in scenarios where the imaging device moves/stops the scanner (ultrasound probe, etc.), the patient moves/stops, and the system is used in imaging devices equipped with peripherals with different processing speeds.

### Problems to Be Solved by the Invention

The object of the present invention is to create a method/system that can adapt the markings of deep learning systems that mark images created with methods (especially ultrasonographic or elastographic methods) used for obtaining anatomical images for medical purposes in accordance with the movement, change and changes in processing speed in the image and can increase the clinical stability of markings made within the deep learning system.

The system has been optimized to provide operation in devices with low processing power capacity (equal and constant in devices with low and high processing power capacity systems) with the invention. More stable responses will be obtained from medical imaging devices equipped with peripherals with different technical features in this way.

Similarly, the reactions and visual results of the movement speeds and changes based on the movement of the scanner and the patient's movements of the imaging device on the deep learning systems can be stabilized with the system of the invention.

Deep learning systems that can be operated with maximum performance with low processor use and mark the images created with medical anatomical image acquisition methods can be put forward thanks to the stabilization of the operation of the system.

The embodiment of the invention is operated by taking the continuous average of neural network responses. Errors such as sudden changes in pure estimates and displacement of structures were tried to be prevented in this way.

### DESCRIPTION OF THE FIGURES

FIG. 1. A schematic view of the inventive method

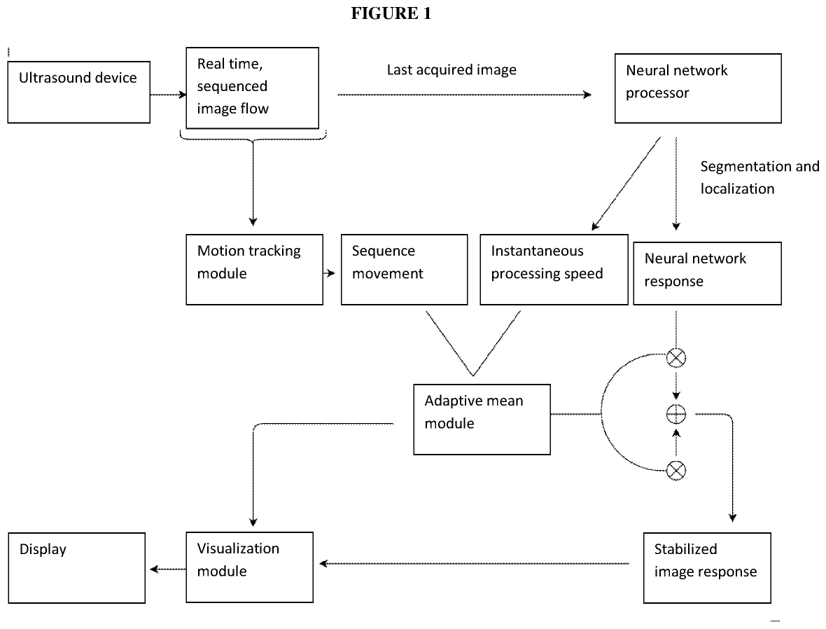

### DESCRIPTION OF THE INVENTION

The invention relates to at least one application that can take an image of the at least one anatomical structure and the surrounding tissue, comprises convolutional neural networks to automatically detect the anatomical object and the surrounding tissue, provides automatic localization and segmentation of the tissue around the image with the use of convolutional neural networks, can automatically mark the anatomical structure and the surrounding tissue in the created image and can present it to the user as an image, comprises at least one processor on which said application can work, comprises a medical anatomical imaging system that has a display unit to present the created image to the user, provides an image of the anatomical object and the surrounding tissue and can transfer this image to said processor. The anatomical imaging system for medical purposes comprises a classical imaging or deep learning-based motion tracking module that processes the images consecutively in sequences taken from the flowing images and removes their mobility in the image during the change and sequence.

More specifically, this imaging system comprises the following:
selecting the points where the anatomical object in the flowing image and the surrounding tissue can be followed and followed successfully, removing the location of the followed points in the next image, removing the points that cannot be matched to their places in the next image from the selected pool,
comprising at least one part of the matched points that has a motion tracking module that allows the amount of change between the images to be found by averaging between the motion amounts.

Errors such as sudden changes in pure estimates and displacement of structures were tried to be prevented by ensuring that neural network responses were constantly averaged in this way. The motion tracking module creates the amount of change between each consecutive image frame that changes within the flowing image by averaging the matching distance and/or movement of the matched points.

Said module ensures that the system regularly follows the movement by following the matched points in the next image. When the number of active points tracked drops below a certain number as a result of the points leaving the screen or lack of successful tracking, the point is selected again.

According to the preferred embodiment of the invention, the motion tracking module will be able to regulate the response on the display unit of the imaging device by changing the reminder factor during the use of the average taken between the motion amount of at least one part of the matched points according to the movement, change and changes in the processing speed.

The effect of the new response on the imaging device elements decreases and constant reactions may occur as the reminder factor increases in this context.

The effect of the new response on the imaging device elements increases when the reminder factor is reduced and a real-time response that matches and follows the anatomical structures can be created.

The average used is far from the movement and location of the anatomical structures on the screen when the change and movement in the image is large (a device that can provide high FPS, moving the probe used for imaging purposes, etc.). The reminder factor is reduced by the tracking module of the invention and the adjustment is made after the elements on the screen show the new reaction in this case.

The need to update the structures in the flowing image will decrease if there is no change in the flowing image (the probe is fixed in a certain position) and the movement is low. The elements shown in the display unit are expected to be stable to form trust under this condition, and this expectation-end tracking module will be able to increase the reminder factor.

The reminder factor cannot be operated stationary since the tracking module aims to work in the flowing image. The tracking module will be able to adaptively change the reminder factor with the change in the image.

The elements on the screen may be behind the ultrasound image if the reminder factor value is high since the reminder factor determines the degree of predicting the unmatched values using the average. This may cause an inconsistent display for the user.

Choosing a low reminder value may prevent the achievement of stability, which is important for clinical confidence. Small changes may cause large response changes in this case.

Therefore, continuous regulation of the reminder value by a tracking module will be important in terms of imaging stability.

The measured movement and reminder factor values used are also affected by the FPS (frames per second) value. The tracking module can regulate the reminder factor depending on the processing power of the system where the product works.

The increase in the time between this processed image requires the reminder factor values used in the product to change, otherwise the reactions may be slow to update or may lose their stability by updating too quickly.

The tracking module can be preset and operated in accordance with the predetermined reminder factors for imaging devices with different peripherals with different processing power according to one of the embodiments of the invention.

The formula presented below can be used to regulate the reminder factor (RF) under changing conditions according to one of the embodiments of the invention.
(RF)*mean+(1−RF)*instant response

The anatomical imaging method for medical purposes of the invention, in its most basic form, comprises the following steps:
providing at least one application that can take an image of at least one anatomical structure and surrounding tissue, includes convolutional neural networks to automatically detect the anatomical object and surrounding tissue, provides automatic localization and segmentation of the anatomical structures that appear on the single image and the surrounding tissue of the image by using convolutional neural networks, can automatically mark the anatomical structure and surrounding tissue in the generated image and present it to the user as an image;
providing at least one processor on which the said application can run;
developing and training a deep learning network comprising one or more convolutional neural networks to automatically detect the anatomical object and surrounding tissue;
automatically positioning and segmenting the anatomical object and surrounding tissue of the image through the neural network; automatically labeling the anatomical object and surrounding tissue in the image;
displaying the labeled image to a user.

Said method comprises the following process steps;
converting the output of the classical imaging or deep learning-based motion tracking module, which processes the images consecutively in the sequences taken from the flowing images and removes the mobility in the image during the change and sequence, into a reminder factor (RF) according to the instantaneous processing speed and creating the visualization from the reaction processed with the reminder factor.

More specifically, said system comprises the following steps:
selecting the points on the flowing image where the anatomical object and the surrounding tissue can be identified and successfully tracked,
removing the location of the followed points in the next image, removing the points that cannot be matched to their places in the next image from the selected pool,
finding the amount of change between the images by averaging the amount of movement of at least a part of the matched points.

More particularly, said method may comprise:
placing it at points that cannot be matched to each new incoming image frame using the points matched in the image frames in the flowing image,
the system regularly following the movement by following them in the next image,
selecting the point again when the number of active points tracked drops below a certain number as a result of the points leaving the screen or the absence of successful tracking.

Instead of using points in an alternative embodiment of the inventive method, the mobility result during the sequence can be inferred by taking direct sequence input using a deep learning network.

According to the preferred embodiment of the method of the invention, it comprises the process step of regulating the response on the display unit of the imaging device by changing the reminder factor during the use of the average taken between the motion amount of at least one part of the matched points according to the movement, change and changes in the processing speed.

1. An anatomical imaging system for medical purposes that enables the generation of an image of the anatomical object and the surrounding tissue and can transfer this image to the said processor, comprising a least one application that can take an image of at least one anatomical structure and surrounding tissue, that includes convolutional neural networks to automatically detect the anatomical object and surrounding tissue, that provides automatic localization and segmentation of the anatomical structures that appear on the image and the surrounding tissue of the image by using convolutional neural networks, that can automatically mark the anatomical structure and surrounding tissue in the generated image and present it to the user as an image; at least one processor on which the said application can run; a display unit to present the created image to the user; characterized in that it comprises a classical imaging or deep learning-based motion tracking module that processes the images consecutively in the flowing sequences to prevent errors such as sudden changes in pure predictions and displacement of the structures by providing a continuous average of the neural network responses.

2. An anatomical imaging system according to claim 1, characterized in that it comprises classical imaging or deep learning based motion tracking module that can select the points in the flowing image that can be tracked successfully and can be tracked successfully belonging to the anatomical object and the surrounding tissue, that enables the location of the tracked points in the next image to be extracted, that enables the removal of the points that cannot be matched to their places in the next image from the selected pool, and that enables the amount of change between the images to be found by averaging the amount of movement of at least one part of the matched points between them, and that extracts the mobility in the image.

3. An anatomical imaging system for medical purposes, comprising the process steps of Providing at least one application that can take an image of at least one anatomical structure and surrounding tissue, that includes convolutional neural networks to automatically detect the anatomical object and surrounding tissue, that provides automatic localization and segmentation of the anatomical structures that appear on the single image and the surrounding tissue of the image by using convolutional neural networks, that can automatically mark the anatomical structure and surrounding tissue in the generated image and present it to the user as an image; providing at least one processor on which the said application can run; developing and training a deep learning network comprising one or more convolutional neural networks to automatically detect the anatomical object and surrounding tissue; automatically positioning and segmenting the anatomical object and surrounding tissue of the image through the neural network; automatically labeling the anatomical object and surrounding tissue in the image; displaying the labeled image to a user; characterized in that it comprises the process steps of converting the output of the classical vision or deep learning based motion tracking module, which extracts the change in the image and the mobility in the image during the sequence by sequentially processing the images in sequences taken from the flowing images, into a recall factor (RF) according to the instantaneous processing speed and creating the visualization from the response processed with the recall factor

4. An anatomical imaging method for medical purposes according claim 3, characterized in that it comprises the following steps:
selecting the points on the flowing image where the anatomical object and the surrounding tissue can be identified and successfully tracked,
removing the location of the followed points in the next image,
removing the points that cannot be matched to their places in the next image from the selected pool,
finding the amount of change between the images by averaging the amount of movement of at least a part of the matched points.

5. An anatomical imaging method for medical purposes according to claim 4, characterized in that it comprises the following steps:
using the points matched in the image frames in the flowing image, placing the points that cannot be matched in each new incoming image frame, following them in the incoming image, the system regularly follows the movement, and when the number of active points followed falls below a certain number as a result of the points leaving the screen or no successful tracking, selecting points again, regulating the response on the display unit of the imaging device by changing the reminder factor during the use of the average taken between the motion amount of at least one part of the matched points according to the movement, change and changes in the processing speed, reducing the reminder factor so that the elements on the screen show the new reaction when the change and movement in the image is large, increasing the reminder factor in the event that there is no change in the flowing image and the movement is low.

6. (canceled)

7. (canceled)

8. An anatomical imaging method for medical purposes according to claim 5, characterized in that it comprises the process step of regulating the reminder factor depending on the processing power of the system it works with.

9. An anatomical imaging method for medical purposes according to claim 6, characterized in that it comprises the process step of presetting the tracking module in accordance with the predetermined reminder factors for imaging devices with different processing power.

10. An anatomical imaging method for medical purposes according to claim 5, characterized in that it comprises the process step of using (RF)*mean+(1−RF)*instant response so that the reminder factor (RF) can be regulated under changing conditions.
